(19) United States
(12) Patent Application Publication
(10) Pub. No.: US 2025/0342965 A1
(43) Pub. Date: Nov. 6, 2025

(54) METHOD AND APPARATUS FOR PREDICTING PROGRESSION TRAJECTORY FROM ACUTE KIDNEY INJURY (AKI) TO KIDNEY DISEASES

(71) Applicants: TAIPEI MEDICAL UNIVERSITY, Taipei City (TW); NATIONAL YANG MING CHIAO TUNG UNIVERSITY, Hsinchu (TW)

(72) Inventors: Shinn-Ying HO, Hsinchu (TW); Mai-Szu WU, Taipei City (TW); Mei-Yi WU, Taipei City (TW); Yu-Wei CHEN, Taipei City (TW); Chun-Hsien CHEN, Hsinchu (TW); Ling-Zi CAO, Hsinchu (TW); Cheng-Yeh LIN, Hsinchu (TW)

(21) Appl. No.: 18/654,517

(22) Filed: May 3, 2024

(51) Int. Cl. G16H 50/30; G16H 50/20

(52) U.S. Cl. CPC G16H 50/30; G16H 50/20

(57) ABSTRACT

The present disclosure provides a method and an apparatus for predicting a progression trajectory from acute kidney injury (AKI) to a kidney disease. The method includes the following steps: receiving a first set of features of a particular acute kidney injury (AKI) patient; selecting a second set of features from the first set of features using a preset algorithm; and predicting, using a first machine-learning model, a progression trajectory of a kidney disease of the particular AKI patient based on the second set of features.

### FIELD OF THE INVENTION

The present disclosure relates to prediction of kidney diseases, and, in particular, to a method and an apparatus for predicting a progression trajectory from acute kidney injury (AKI) to kidney diseases.

### BACKGROUND

The treatment strategies of patients diagnosed with acute kidney injury (AKI) are dependent on potential risk of progression to acute kidney disease (AKD), chronic kidney disease (CKD), and end stage kidney disease (ESKD). Patients who experience acute kidney injury (AKI) are at risk of developing acute kidney disease (AKD), chronic kidney disease (CKD), and end stage kidney disease (ESKD). Failure to intervene in a timely manner may result in the development of end stage kidney disease (ESKD) and necessitate renal replacement therapy (RRT), such as hemodialysis. Compared to patients without AKI, those with AKI face a higher likelihood of developing CKD, end-stage kidney disease (ESKD), and other unfavorable outcomes. If it were possible to predict the development of AKD, CKD, and ESKD in AKI patients, it would enable early identification of the trajectory from AKI to AKD, CKD, and ESKD, allowing for intervention to prevent further progression. Consequently, there is a need for a method and apparatus capable of predicting the progression trajectory from AKI to kidney diseases such as AKD, CKD, and ESKD.

### SUMMARY OF THE DISCLOSURE

In an aspect of the present disclosure, a method for predicting a progression trajectory from acute kidney injury (AKI) to a kidney disease is provided. The method includes the following steps: receiving a first set of features of a particular acute kidney injury (AKI) patient; selecting a second set of features from the first set of features using a preset algorithm; and predicting, using a first machine-learning model, a progression trajectory of a kidney disease of the particular AKI patient based on the second set of features.

In another aspect of the present disclosure, an apparatus for predicting a progression trajectory from acute kidney injury (AKI) to a kidney disease is provided. The apparatus includes: at least one memory having computer executable instructions stored therein; and at least one processor coupled to the at least one memory. The computer executable instructions cause the at least one processor to perform operations, and the operations includes: receiving a first set of features of a particular acute kidney injury (AKI) patient; selecting a second set of features from the first set of features using a preset algorithm; and predicting, using a first machine-learning model, a progression trajectory of a kidney disease of the particular AKI patient based on the second set of features.

### BRIEF DESCRIPTION OF THE DRAWINGS

Aspects of the present disclosure are best understood from the following detailed description when read with the accompanying figures. It is noted that in accordance with the standard practice in the industry, various features are not drawn to scale. In fact, the dimensions of the various features may be arbitrarily increased or reduced for clarity of discussion.

FIG. 1 is a flowchart of sample pre-processing and labeling of AKI, AKD, CKD, and ESKD in accordance with an embodiment of the present disclosure.

FIG. 2 is a diagram illustrating the timeline of diagnosis of AKI, AKD, and CKD in accordance with an embodiment of the present disclosure.

FIG. 3 is a diagram illustrating a timeline of diagnosis of ESKD in accordance with an embodiment of the present disclosure.

FIG. 4 is a diagram illustrating a timeline of diagnosis of ESKD in accordance with another embodiment of the present disclosure.

FIGS. 5A and 5B are portions of a flowchart of a machine-learning method for predicting a progression trajectory from AKI to AKD, CKD, and ESKD in accordance with an embodiment of the present disclosure.

FIG. 6 is a flowchart of the procedure in block 520 in FIG. 5A.

FIG. 7 is a flowchart of a method for predicting a progression trajectory from acute kidney injury (AKI) to a kidney disease.

FIG. 8 is a schematic diagram showing a computer device 800 according to some embodiments of the present disclosure.

Corresponding numerals and symbols in the different figures generally refer to corresponding parts unless otherwise indicated. The figures are drawn to clearly illustrate the relevant aspects of the various embodiments and are not necessarily drawn to scale.

### DETAILED DESCRIPTION

The following disclosure provides many different embodiments, or examples, for implementing different features of the provided subject matter. Specific examples of operations, components, and arrangements are described below to simplify the present disclosure. These are, of course, merely examples and are not intended to be limiting. For example, a first operation performed before or after a second operation in the description may include embodiments in which the first and second operations are performed together, and may also include embodiments in which additional operations may be performed between the first and second operations. For example, the formation of a first feature over, on or in a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Time relative terms, such as “prior to,” “before,” “posterior to,” “after” and the like, may be used herein for ease of description to describe one operations or feature's relationship to another operation(s) or feature(s) as illustrated in the figures. The time relative terms are intended to encompass different sequences of the operations depicted in the figures. Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Relative terms for connections, such as “connect,” “connected,” “connection,” “couple,” “coupled,” “in communication,” and the like, may be used herein for ease of description to describe an operational connection, coupling, or linking one between two elements or features. The relative terms for connections are intended to encompass different connections, coupling, or linking of the devices or components. The devices or components may be directly or indirectly connected, coupled, or linked to one another through, for example, another set of components. The devices or components may be wired and/or wireless connected, coupled, or linked with each other.

As used herein, the singular terms “a,” “an,” and “the” may include plural referents unless the context clearly indicates otherwise. For example, reference to a device may include multiple devices unless the context clearly indicates otherwise. The terms “comprising” and “including” may indicate the existences of the described features, integers, steps, operations, elements, and/or components, but may not exclude the existences of combinations of one or more of the features, integers, steps, operations, elements, and/or components. The term “and/or” may include any or all combinations of one or more listed items.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

The nature and use of the embodiments are discussed in detail as follows. It should be appreciated, however, that the present disclosure provides many applicable inventive concepts that can be embodied in a wide variety of specific contexts. The specific embodiments discussed are merely illustrative of specific ways to embody and use the disclosure, without limiting the scope thereof.

The increase of incidence and prevalence of acute kidney injury (AKI) is an emerging global health care problem. AKI can lead to acute kidney disease (AKD) and chronic kidney disease (CKD), which is the emerging, top-ranked non-communicable disease causing disabled adjusted life years (DALYs) and enormous economic burden on health care systems. It is estimated that 2 million and 1.2 million people worldwide die each year from AKI and CKD, respectively. Early diagnosis of AKI, AKD and CKD and timely intervention to ameliorate kidney diseases remain a critical unmet medical need.

AKI, AKD, and CKD can be seen as continuous processes. The initial kidney damage can lead to persistent pathological changes that eventually evolves into CKD. The Kidney Disease: Improving Global Outcomes (KDIGO) guideline defined AKI as abrupt deterioration in renal functions in 7 days or less. CKD was defined as abnormal kidney structure or functions for more than 90 days. Recently, consensus has been formed on the definition of AKD, either the persistent renal impairment between 7 and 90 days after the occurrence of AKI or the transitional kidney disease status approaching CKD.

Compared to patients without acute kidney injury (AKI), those with AKI face a heightened risk of developing acute kidney disease (AKD), chronic kidney disease (CKD), end-stage kidney disease (ESKD), and other adverse outcomes. It has been observed that recovering from AKI is linked to a reduced risk of ESKD. If it were possible to predict the likelihood of AKD, CKD, and ESKD development in AKI patients beforehand, it would enable the determination of AKI-AKD-CKD-ESKD progression trajectories and facilitate timely intervention to halt AKI progression.

Accordingly, a machine-learning method for predicting AKI-AKD-CKD-ESKD progression trajectories of AKI patients is proposed in the present disclosure. The proposed method aims to identify a minimal set of risk factors and maximize the prediction accuracy by simultaneous optimization of feature selection and parameter setting of SVM. Instant and precise prediction of AKI-AKD-CKD-ESKD trajectories with modifiable personal risk factors can advance patient-specific preventive, diagnostic, and treatment strategies.

The present disclosure utilized the laboratory and administrative datasets obtained from the health information system of a single tertiary referral medical center to identify a small set of risk factors for predicting progression of AKI patients. The comprehensive dataset comprises multiple-type information, including patient demographics, hospitalized data, ICD-9/ICD-10 codes, emergency records, sequential laboratory values, records of all medication use, etc.

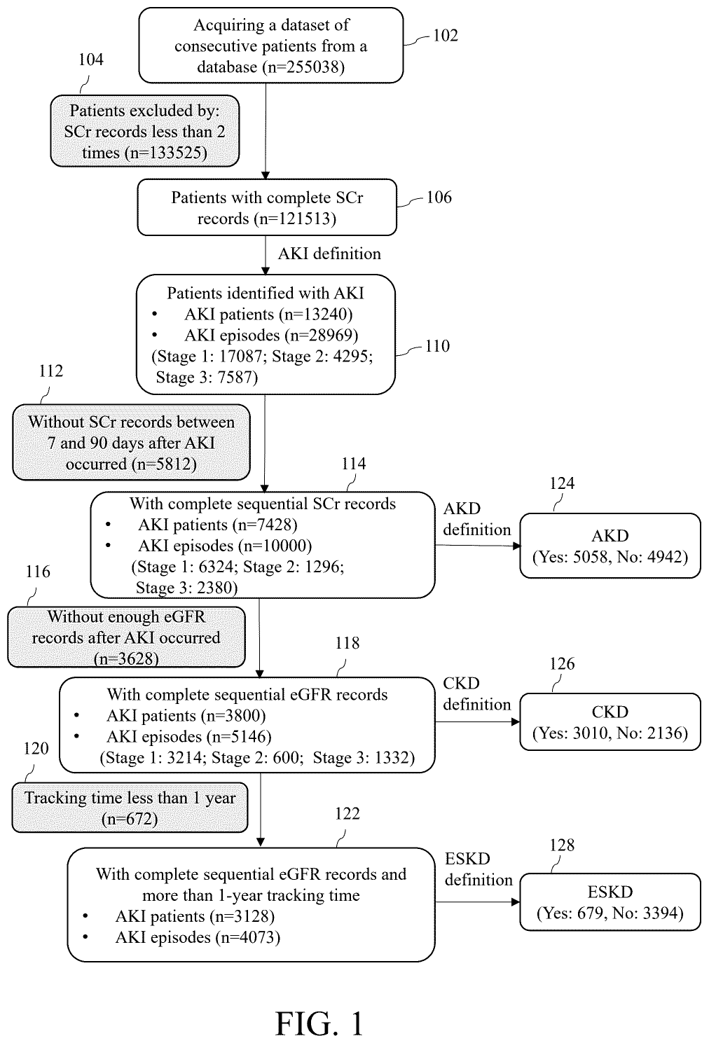

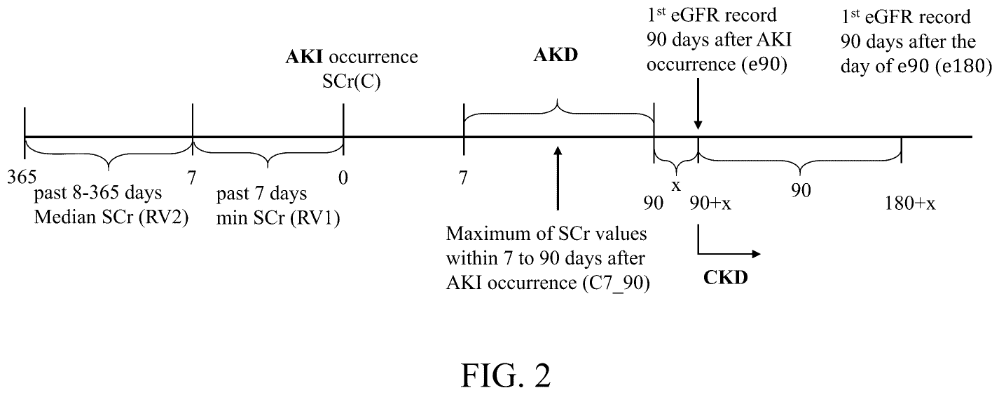

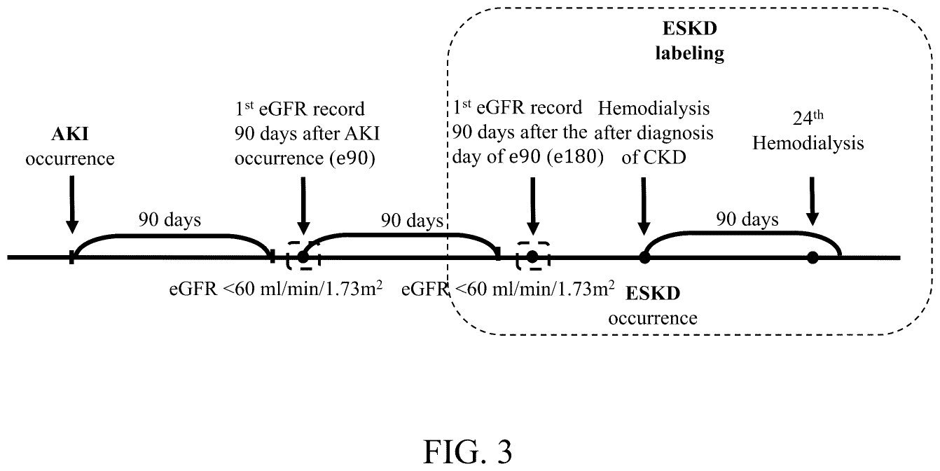

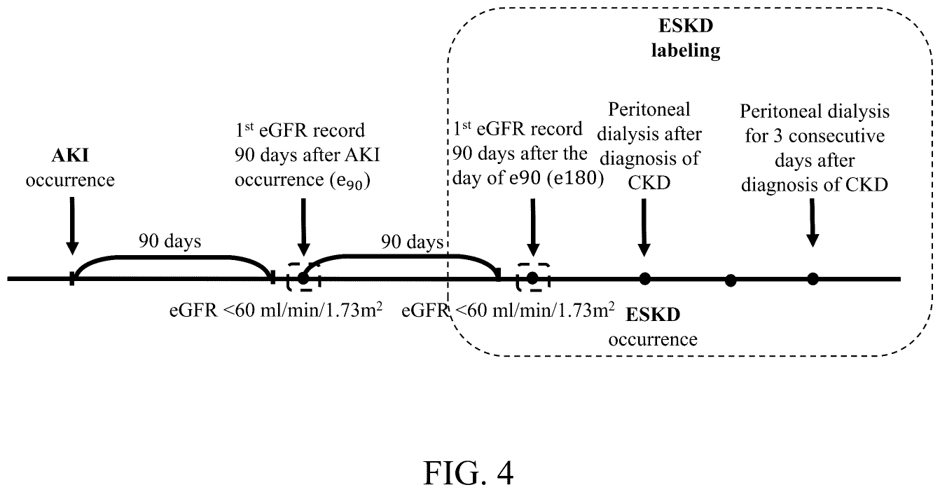

FIG. 1 is a flowchart of sample pre-processing and labeling of AKI, AKD, CKD, and ESKD in accordance with an embodiment of the present disclosure. FIG. 2 is a diagram illustrating the timeline of diagnosis of CKD in accordance with an embodiment of the present disclosure. FIG. 3 is a diagram illustrating a timeline of diagnosis of ESKD in accordance with an embodiment of the present disclosure. FIG. 4 is a diagram illustrating a timeline of diagnosis of ESKD in accordance with another embodiment of the present disclosure.

In some embodiments, a comprehensive dataset, which records from 255,038 (n=255,038) consecutive patients with laboratory values, is acquired from the Shuang-Ho Hospital database (block 102). This dataset encompasses a wide range of information, such as patient demographics, hospitalized data, ICD-9/ICD-10 codes, emergency records, sequential laboratory values, records of all medication use, etc. Subsequently, patients with AKI, CKD, AKD, and ESKD are appropriately identified and labeled using the acquired dataset. For example, the AKI and CKD patients are labeled based on the Kidney Disease: Improving Global Outcomes (KDIGO) guidelines. The AKD patients are labeled according to the consensus definition established by 16th Acute Disease Quality Initiative. As for the ESKD patients, their labeling is determined by the processes outlined in the embodiments of FIGS. 3 and 4, which involves hemodialysis and peritoneal dialysis following the diagnosis of CKD in a given patient.

In some embodiments, due to the loss of follow-up or insufficient serum creatinine (SCr) records to confirm the AKI patients, the patients with less than two SCr records were excluded (n=133,525) (block 104), and the patients with complete SCr records are kept (n=121513) (block 106). As depicted in FIG. 2, to screen the AKI patients, the nadir SCr value within 7 days (RV1) prior to index SCr (C) is used as the baseline value for comparison. If such a SCr value (RV1) was not available, the median value of SCr within the past 8-365 days was used as the surrogate baseline SCr (RV2). Additionally, the AKI episodes over 90 days after AKI occurrence in the same patient as different AKI samples. Using the AKI definition, 28,969 AKI episodes from 13,240 AKI patients were identified (block 110), including 17,087 stage-1 AKI episodes, 4,295 stage-2 AKI episodes, and 7,587 stage-3 AKI episodes. Additionally, 5,812 patients without SCr records between 7 and 90 days after AKI occurrence were excluded (block 112). Finally, 10,000 AKI episodes from 7,428 AKI patients with complete sequential creatinine records were enrolled (block 114).

In some embodiments, the maximum value of the serum creatinine (SCr) values within 7 to 90 days following the occurrence of AKI (C7_90) was retrieved and compared with RV1 and RV2. If the ratio of C7_90/RV1 or C7_90/RV2 exceeded 1.5, the occurrence of AKD would be identified and labeled. Out of the 10,000 AKI samples, 5,058 (50.5%) developed AKD within 7 to 90 days after AKI occurrence (block 124). Additionally, the initial estimated glomerular filtration rate (eGFR) test values within 90 days after AKI occurrence (e90) and the first eGFR test values within 90 days after the day of the initial eGFR test value occurred (e180) were examined. If both e90 and e180 were less than 60 ml/min/1.73 m², the AKI patients would be labeled as progression to CKD. Out of the 10,000 AKI samples, 5,146 AKI samples with complete sequential eGFR records were eligible for classification as CKD or not (block 118), as the remaining 4,854 samples were excluded due to incomplete eGFR records. Finally, 3,010 (58.5%) of the 5146 AKI samples were labeled as CKD after AKI using the aforementioned AKD definition (block 126).

In some embodiments, a total of 672 patients with complete sequential eGFR records and a tracking time less than 1 year were excluded, resulting in 4,073 AKI episodes from 3,128 AKI patients with complete SCr records for more than 1 year being included for ESKD diagnosis. Finally, 679 (16.67%) of the 4073 AKI samples were labeled as ESKD after AKI using the definitions described in the embodiments of FIGS. 3 and 4.

In some embodiments, either the flow in FIG. 3 or that in FIG. 4 can be used to determine whether a given AKI sample can be classified as ESKD. Referring to FIG. 3, the flow shown in FIG. 3 may involve the administration of hemodialysis after the diagnosis of CKD in a given AKI patient. For example, assuming that a given patient has been diagnosed with CKD (e.g., both the first eGFR value (e90) after 90 days of AKI and the first eGFR value (e180) after an additional 90 days are both below 60 mL/min/1.73 m²), when the patient has started hemodialysis (HD) within 90 days after e 180 for a total of 24 times or more, it is defined as the occurrence of end-stage kidney disease (ESKD).

Referring to FIG. 4, the flow shown in FIG. 4 may involve the administration of peritoneal dialysis after the diagnosis of CKD in a given AKI patient. For example, Assuming that a given patient is CKD diagnosed, (e.g., the first eGFR value (e90) after 90 days of AKI and the first eGFR value (e180) after another 90 days are both below 60 mL/min/1.73 m²), when the given patient has undergone peritoneal dialysis (PD) for at least 3 consecutive days, it is defined as the occurrence of end-stage kidney disease (ESKD).

In some embodiments, after labeling the AKI patients with progression to AKD, CKD, and ESKD, 106 raw features (or factors) from five tables may be combined in the comprehensive database. The features of patient's demographics may be excluded by the following criteria: 1) duplicated feature (k=9), 2) irrelevant to this disclosure (k=41), and 3) ICD code (k=3). The laboratory values were selected by the ratio of missing values <50% (k=12). The medication use histories were categorized into 25 types of drugs and 11 of them were adopted by using domain knowledge.

In some embodiments, the following baseline characteristics of 10000 AKI samples may be collected for the candidate feature set shown in Table 1, which may include AKI stage, demographic factors (age, sex, blood type [A, B, AB, and O], drug allergy, and critical illness), laboratory values (SCr, BUN, eGFR, Na, K, GPT, GOT, and white blood cell differential count [Neutrophil, Lymphocyte, Monocyte, Eosinophil, and Basophil]), and medication use history within one month prior to AKI occurrence. The features of the medication use history may be as follows: angiotensin-converting enzyme inhibitor (ACEI), antibiotics, anticholinergics, antifungal, antihypertensive, antiviral, chemotherapy, diuretics, sodium glucose cotransporters 2 inhibitors (SGLT2i), non-steroidal anti-inflammatory (NSAID), and proton pump inhibitor (PPI). Several features were additionally derived from the available features. To find out the effect of drug combination on the AKI-AKD-CKD trajectories, 17 features may be generated by various combinations of antibiotics, antifungal, antihypertensive, chemotherapy, diuretics, NSAID, and PPI. Totally, 55 candidate features were initially used for ELAKI to identify the risk factors of the progression to AKD, CKD, and ESKD. It should be noted that the present disclosure is not limited to the aforementioned 55 candidate features, and one or more candidate features can be added in some embodiments.

TABLE 1

| | | | KDIGO AKI stage | | | | | | |
|---|---|---|---|---|---|---|---|---|---|
| Characteristics | All, n = 10000 | | Stage1, n = 6324 | | Stage2, n = 1296 | | Stage3, n = 2380 | | p-value |
| Demographics | | | | | | | | | |
| Age, yr, median (IQR) | 70 | (60-80) | 70 | (60-80) | 71 | (60-82) | 68 | (59-79) | 0.002 |
| Men, n (%) | 5285 | (53) | 3312 | (52) | 650 | (50) | 1323 | (56) | 0.003 |
| Blood type, n (%) | | | | | | | | | |
| A | 2195 | (22) | 1384 | (22) | 264 | (20) | 547 | (23) | 0.09 |
| B | 2021 | (20) | 1172 | (19) | 265 | (20) | 584 | (25) | 0.003 |
| AB | 465 | (5) | 284 | (4) | 60 | (5) | 121 | (5) | 0.98 |
| O | 3650 | (37) | 2249 | (36) | 463 | (36) | 938 | (39) | 0.56 |
| Drug allergy, n (%) | 7554 | (76) | 4736 | (75) | 981 | (76) | 1837 | (77) | 0.08 |
| Critical illness, n (%) | 6651 | (67) | 4122 | (65) | 866 | (67) | 1663 | (70) | 0.18 |
| Laboratory values, | | | | | | | | | |
| median (IQR) | | | | | | | | | |
| Creatinine, mg/dl | 2.1 | (1.4-4.0) | 1.6 | (1.2-2.4) | 2.1 | (1.6-2.7) | 6.8 | (4.8-9.7) | <0.001 |
| BUN, mg/dl | 41 | (24-69) | 31 | (20-49) | 38 | (24-57) | 76 | (55-102) | <0.001 |
| eGFR, ml/min per 1.73 m² | 29.2 | (13.2-47.8) | 39.6 | (25.0-56.7) | 29.7 | (21.7-40.6) | 7.5 | (5.1-11.4) | <0.001 |
| Na, mmol/L | 137 | (133-140) | 137 | (134-140) | 136 | (132-140) | 136 | (133-139) | <0.001 |
| K, mmol/L | 4.1 | (3.6-4.6) | 4 | (3.5-4.5) | 4.1 | (3.5-4.7) | 4.3 | (3.7-5) | <0.001 |
| GPT, IU/L | 21 | (15-35) | 22 | (15-35) | 25 | (16-48) | 17 | (13-27) | 0.009 |
| GOT, IU/L | 28 | (21-44) | 29 | (21-45) | 31 | (23-52) | 24 | (18-37) | 0.415 |
| WBC differential count | | | | | | | | | |
| Neutrophil, % | 0.78 | (0.68-0.86) | 0.77 | (0.67-0.86) | 0.79 | (0.70-0.87) | 0.78 | (0.69-0.85) | <0.001 |
| Lymphocyte, % | 0.11 | (0.06-0.19) | 0.12 | (0.06-0.20) | 0.10 | (0.05-0.17) | 0.11 | (0.06-0.17) | <0.001 |
| Monocyte, % | 0.07 | (0.05-0.10) | 0.07 | (0.05-0.10) | 0.07 | (0.042-0.094) | 0.07 | (0.05-0.10) | 0.144 |
| Eosinophil, % | 0.01 | (0.00-0.02) | 0.01 | (0.00-0.02) | 0.003 | (0-0.012) | 0.01 | (0.001-0.03) | <0.001 |
| Basophil, % | 0.003 | (0.001-0.006) | 0.003 | (0.001-0.006) | 0.003 | (0.00-0.005) | 0.004 | (0.001-0.007) | <0.001 |
| Medication use, n (%) | | | | | | | | | |
| ACEI | 1198 | (12) | 831 | (13) | 142 | (11) | 225 | (9) | <0.001 |
| Antibiotics | 4528 | (45) | 2986 | (47) | 668 | (52) | 874 | (37) | <0.001 |
| Anticholinergic drug | 462 | (5) | 298 | (5) | 64 | (5) | 100 | (4) | 0.348 |
| Antifungal drug | 141 | (1) | 94 | (1) | 22 | (2) | 25 | (1) | 0.058 |
| Antihypertensive drug | 1745 | (17) | 1056 | (17) | 181 | (14) | 508 | (21) | <0.001 |
| Antiviral drug | 77 | (1) | 49 | (1) | 17 | (1) | 11 | (0) | 0.023 |
| Chemotherapy | 371 | (4) | 285 | (5) | 50 | (4) | 36 | (2) | <0.001 |
| Diuretics | 2445 | (24) | 1627 | (26) | 308 | (24) | 510 | (21) | <0.001 |
| SGLT2i | 52 | (1) | 45 | (1) | 6 | (0) | 1 | (0) | 0.004 |
| NSAID | 398 | (4) | 275 | (4) | 78 | (6) | 45 | (2) | <0.001 |
| PPI | 2291 | (23) | 1526 | (24) | 304 | (23) | 461 | (19) | <0.001 |

IQR, interquartile range;
BUN, blood urea nitrogen;
GPT, glutamic pyruvic transaminase;
GOT, glutamic oxaloacetic transaminase;
WBC, white blood cell;
ACEI, angiotensin converting enzyme inhibitor;
SGLT2i, sodium glucose cotransporters 2 inhibitors;
NSAID, non-steroidal anti-inflammatory;
PPI, proton pump inhibitor;
KDIGO, Kidney Disease Improving Global Outcomes.

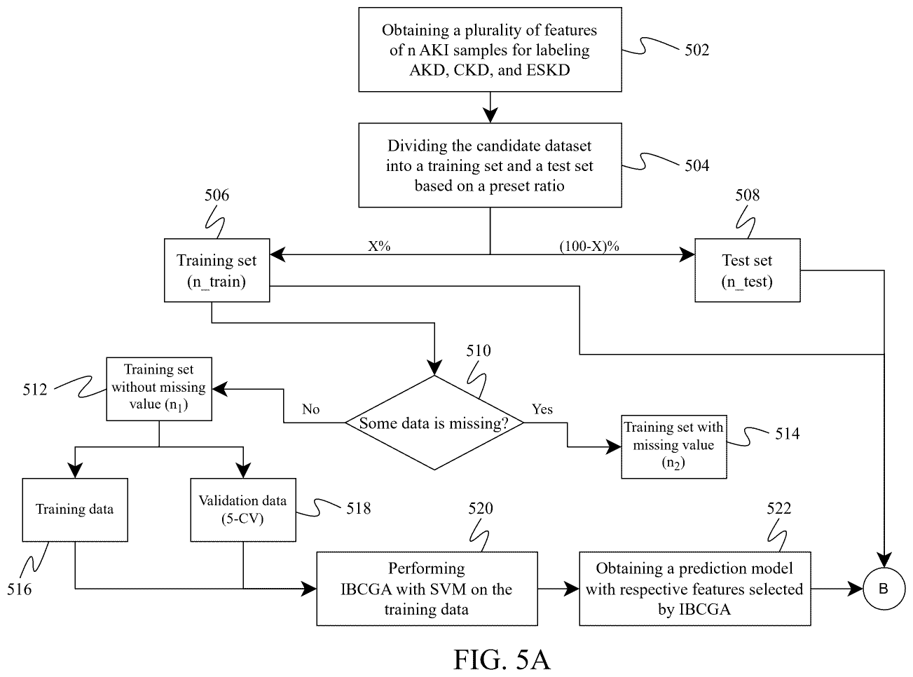

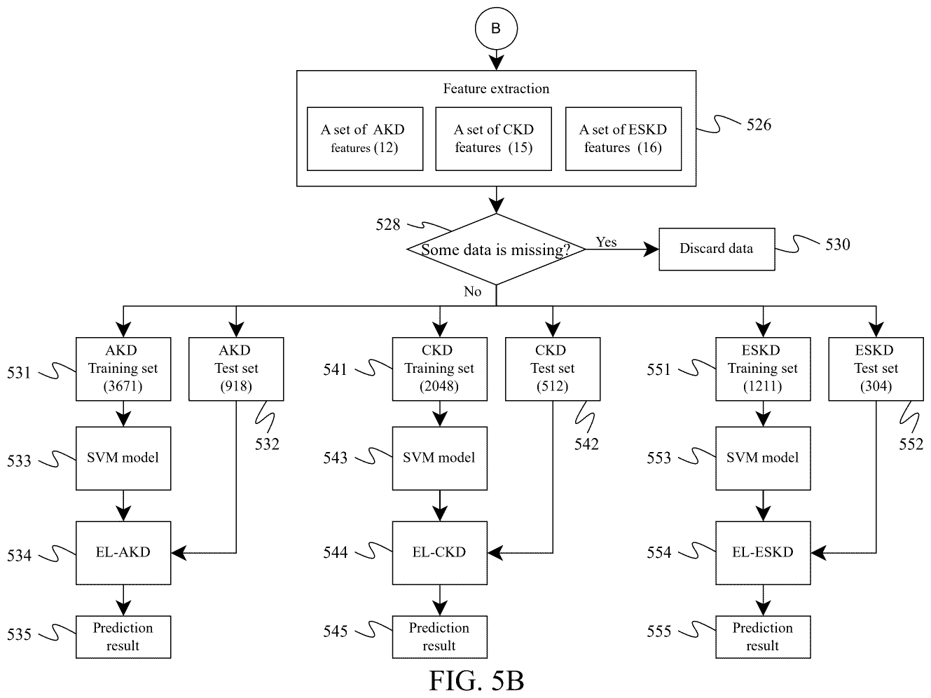

FIGS. 5A and 5B are portions of a flowchart of a machine-learning method for predicting a progression trajectory from AKI to AKD, CKD, and ESKD in accordance with an embodiment of the present disclosure.

In some embodiments, method 500 shown in FIGS. 5A-5B, referred to as ELAKI, may be a novel evolutionary machine-learning method for predicting the risk score of trajectories from AKI to AKD, CKD, and ESKD. ELAKI incorporates demographics, laboratory values, and medication use history into a candidate feature set. Additionally, method 500 utilizes an intelligent genetic algorithm for feature selection in conjunction with a machine learning technique, such as an SVM classifier. The SVM classifier employs a kernel function to map the training data into a higher-dimensional feature space and identifies the hyperplane that maximizes the margin between two classes. Method 500 consists of three stages: (A) a data preprocessing stage, (B) a customized IBCGA (inheritable bi-objective combinatorial genetic algorithm) stage for signature identification, and (C) a training dataset enlarging stage. In some embodiments, method 500 can also utilize any other machine-learning feature-selection algorithm, such as a filter-based technique (e.g., information gain, chi-square test, fisher's score, missing value ratio, etc.), a wrapper-based technique (e.g., forward selection, backward selection, exhaustive feature selection, recursive feature elimination, etc.), and an embedded technique (e.g., regularization, random forest importance, etc.), for feature selection. For purposes of description, the method 500 using the customized IBCGA algorithm with the SVM classifier is described as follows.

The data preprocessing stage may start with block 502. Block 502: Obtaining a candidate dataset including a plurality of candidate features of a plurality of AKI samples. For example, the candidate dataset may include 55 candidate features of the AKI samples (e.g., 10000 AKI samples) shown in Table 1, which includes AKI stage, demographic factors, laboratory values, and medication use history.

Block 504: Dividing the candidate dataset into a training set (e.g., block 506) and a test set (e.g., block 508) based on a preset ratio. For example, n_train and n_test may denote the number of samples in the training set and test set, respectively. The ratio of n_train to n_test may be X %, wherein the value of X can be adjusted according to practical needs. In some embodiments, the value of X is 80.

Block 510: Determining whether some data is missing. For example, in some embodiments, in order to accurately identify the risk factors (e.g., signatures) of progression to AKD, CKD, and ESKD, the first training subset in block 512, which includes AKI samples without missing values in the training set in block 506, may be used as the phase-1 training datasets of ELAKI for AKD (n1=2312), CKD (n1=952), and ESKD (n1=706), respectively. The second training subset in block 514, which includes AKI samples with one or more missing values in the training sets, may not be employed in ELAKI for identifying the risk factors of progression to AKD, CKD, and ESKD.

The customized IBCGA stage with signature may include blocks 516 to 522. In some embodiments, the training set without missing value (block 512) may be divided into training data (block 516) and validation data (block 518). The fitness function of the IBCGA to guide the search for an optimal solution was to maximize the accuracy of predicting AKD, CKD, and ESKD using 5-fold cross-validation (5-CV). For example, in k-fold cross-validation, the original sample is randomly partitioned into k equal sized subsamples, often referred to as “folds”. Of the k subsamples, a single subsample is retained as the validation data for testing the model, and the remaining (k−1) subsamples are used as training data. The cross-validation process is then repeated k times, with each of the k subsamples used exactly once as the validation data. The k results can then be averaged to produce a single estimation. The advantage of this method over repeated random sub-sampling is that all observations are used for both training and validation, and each observation is used for validation exactly once.

Block 520: Performing IBCGA with SVM (ELAKI) on respective training data for predicting AKD, CKD, and ESKD. Subsequently, the ELAKI may output the models EL-AKD, EL-CKD, and EL-ESKD for predicting AKD, CKD, and ESKD, respectively. It should be noted that the models EL-AKD, EL-CKD, and EL-ESKD may be the best prediction models with least features for predicting AKD, CKD, and ESKD, respectively. In some embodiments, ELAKI may identify 12, 15, and 16 features as the signatures to design the EL-AKD, EL-CKD, and EL-ESKD models, respectively.

In some embodiments, the risk factors in the AKD, CKD, and ESKD signatures may be ranked by main effect difference (MED), as shown by Tables 2-1, 2-2, and 2-3, respectively.

TABLE 2-1

| | | | p-value | p-value |
|---|---|---|---|---|
| Rank | AKD features | MED | (Univariate) | (Multivariate) |
| 1 | AKI stage | 0.03 | <0.001 | <0.001 |
| 2 | Diuretics | 0.02 | <0.001 | <0.001 |
| 3 | eGFR | 0.01 | 0.22 | 0.006 |
| 4 | NSAID (injection) | 0.01 | 0.08 | 0.13 |
| 5 | Anti-cholinergic | 0.01 | 0.10 | 0.05 |
| 6 | ACEI | 0.008 | 0.06 | 0.007 |
| 7 | PPI (oral) | 0.007 | 0.05 | 0.28 |
| 8 | Creatinine | 0.006 | 0.32 | 0.03 |
| 9 | BUN | 0.004 | 0.22 | 0.43 |
| 10 | Antibiotics | 0.002 | 0.07 | 0.35 |
| 11 | GPT | 0.001 | 0.58 | 0.39 |
| 12 | Drug allergy | <0.001 | 0.43 | 0.25 |

TABLE 2-2

| | | | p-value | p-value |
|---|---|---|---|---|
| Rank | CKD features | MED | (Univariate) | (Multivariate) |
| 1 | eGFR | 0.13 | <0.001 | <0.001 |
| 2 | Creatinine | 0.10 | <0.001 | <0.001 |
| 3 | GOT | 0.01 | 0.43 | <0.001 |
| 4 | Diuretics (injection) | 0.01 | <0.001 | 0.59 |
| 5 | NSAID | 0.01 | 0.10 | 0.12 |
| 6 | Monocyte | 0.009 | <0.001 | 0.32 |
| 7 | Blood type A | 0.007 | 0.98 | 0.71 |
| 8 | Diuretics | 0.004 | <0.001 | 0.14 |
| 9 | SGLT2i | 0.004 | 0.68 | 0.90 |
| 10 | Antibiotics & Diuretics | 0.003 | 0.18 | 0.008 |
| | & PPI (injection) | | | |
| 11 | Antibiotics (oral) | 0.003 | 0.01 | 0.45 |
| 12 | Drug allergy | <0.001 | 0.001 | 0.77 |
| 13 | Anti-fungal | <0.001 | 0.68 | 0.88 |
| 14 | Basophil | <0.001 | <0.001 | 0.46 |
| 15 | AKI stage | <0.001 | <0.001 | <0.001 |

TABLE 2-3

| | | | p-value | p-value |
|---|---|---|---|---|
| Rank | ESKD features | MED | (Univariate) | (Multivariate) |
| 1 | Creatinine | 0.314 | <0.001 | <0.001 |
| 2 | AKI stage | 0.116 | <0.001 | <0.001 |
| 3 | Basophil | 0.056 | <0.001 | 0.005 |
| 4 | K (category) | 0.034 | 0.812 | <0.001 |
| 5 | Blood type O | 0.029 | 0.839 | 0.844 |
| 6 | Sex | 0.024 | 0.284 | <0.001 |
| 7 | Critical illness | 0.023 | 0.251 | 0.924 |
| 8 | Diuretics | 0.022 | 0.661 | 0.233 |
| 9 | Diuretics (oral) | 0.019 | 0.521 | 0.699 |
| 10 | Diuretics (injection) | 0.006 | 0.111 | 0.781 |
| 11 | GOT (category) | 0.004 | <0.001 | 0.137 |
| 12 | Age | 0.002 | 0.152 | 0.008 |
| 13 | Antibiotics (injection) | <0.001 | <0.001 | 0.054 |
| 14 | Antibiotics | <0.001 | 0.274 | <0.001 |
| 15 | Chemotherapy | <0.001 | 0.267 | 0.334 |
| 16 | BUN | <0.001 | <0.001 | <0.001 |

In some embodiments, based on MED, the risk factors can be ranked according to the prediction contribution. The respective identified signatures for AKD, CKD, and ESKD may consists of a small set of risk factors. Referring to Table 2-1, the top-3 factors of the AKD signature are AKI stage, diuretics usage, and eGFR. Referring to Table 2-2, the top-3 factors of CKD signature are eGFR, SCr, and GOT. Referring to Table 2-3, the top-3 factors of ESKD signature are Creatinine, AKI stage, and Basophil. The common factors between the AKD, CKD, and ESKD signatures may be a value of creatinine, use of diuretics, and use of antibiotics. In some embodiments, when a significant factor (p-value <0.001) has a relatively low rank, e.g., the AKI stage in the CKD signature ranked at 15, it may not be selected in advancing prediction accuracy.

In some embodiments, the training dataset enlarging phase may start with block 526, and the AKD, CKD, and ESKD signatures may be used to re-extract samples from the whole training set (n_train) and test set (n_test) for training AKD, CKD, and ESKD SVM models, respectively. For example, in block 526, respective feature extraction is performed to extract the 12 AKD signatures, 15 CKD signatures, and 16 ESKD signatures obtained from the ELAKI.

In block 528, it is determined whether any data corresponding to the 12 AKD signatures, 15 CKD signatures, and 16 ESKD signatures is missing in each AKI sample. For example, the whole dataset, including the training set (n_train) in block 506 and the test set (n_test) in block 508, may be used to extract the AKI samples with the 12 AKD signatures, 15 CKD signatures, and 16 ESKD signatures to build respective data pools for building the EL-AKD, EL-CKD, and EL-ESKD models. If a particular AKI sample lacks one of the 12 AKD signatures, the particular AKI sample will be discarded from the respective data pool for building the EL-AKD model (block 530). Similar operations can be applied to the AKI samples in the respective data pools for building the EL-CKD and EL-ESKD models.

In some embodiments, the AKI samples within a first data pool for building the EL-AKD model may be divided into an AKD training set (block 531) and an AKD test set (block 532) according to the preset ratio (e.g., 80% to 20%). That is, the AKD training set may include 80% of total AKI samples within the first data pool. For example, the AKD training set may include 3671 AKI samples, while the AKD test set may include 918 AKI samples.

In some embodiments, the AKD training set in block 531 may be used to train a first SVM model (block 533), resulting in a trained SVM model (e.g., EL-AKD model) (block 534). Subsequently, the AKD test set in block 532 can be inputted into the EL-AKD model to generate prediction results (block 535) regarding the progression trajectory from AKI to AKD. Accordingly, the new phase-2 AKD training set and AKD test set may be used to establish and evaluate the EL-AKD model.

In some embodiments, the AKI samples within a second data pool for building the EL-CKD model may be divided into a CKD training set (block 541) and a CKD test set (block 542) according to the preset ratio (e.g., 80% to 20%). That is, the CKD training set may include 80% of total AKI samples within the second data pool. For example, the CKD training set may include 2048 AKI samples, while the AKD test set may include 512 AKI samples.

In some embodiments, the CKD training set in block 541 may be used to train a second SVM model (block 543), resulting in a trained SVM model (e.g., EL-CKD model) (block 544). Subsequently, the CKD test set in block 542 can be inputted into the EL-CKD model to generate prediction results (block 545) regarding the progression trajectory from AKI to CKD. Accordingly, the new phase-2 CKD training set and CKD test set may be used to establish and evaluate the EL-CKD model.

In some embodiments, the AKI samples within a third data pool for building the EL-ESKD model may be divided into an ESKD training set (block 531) and an ESKD test set (block 532) according to the preset ratio (e.g., 80% to 20%). That is, the ESKD training set may include 80% of total AKI samples within the third data pool. For example, the ESKD training set may include 1211 AKI samples, while the ESKD test set may include 304 AKI samples.

In some embodiments, the ESKD training set in block 551 may be used to train a third SVM model (block 553), resulting in a trained SVM model (e.g., EL-ESKD model) (block 554). Subsequently, the ESKD test set in block 552 can be inputted into the EL-ESKD model to generate prediction results (block 555) regarding the progression trajectory from AKI to ESKD. Accordingly, the new phase-2 ESKD training set and ESKD test set may be used to establish and evaluate the EL-ESKD model.

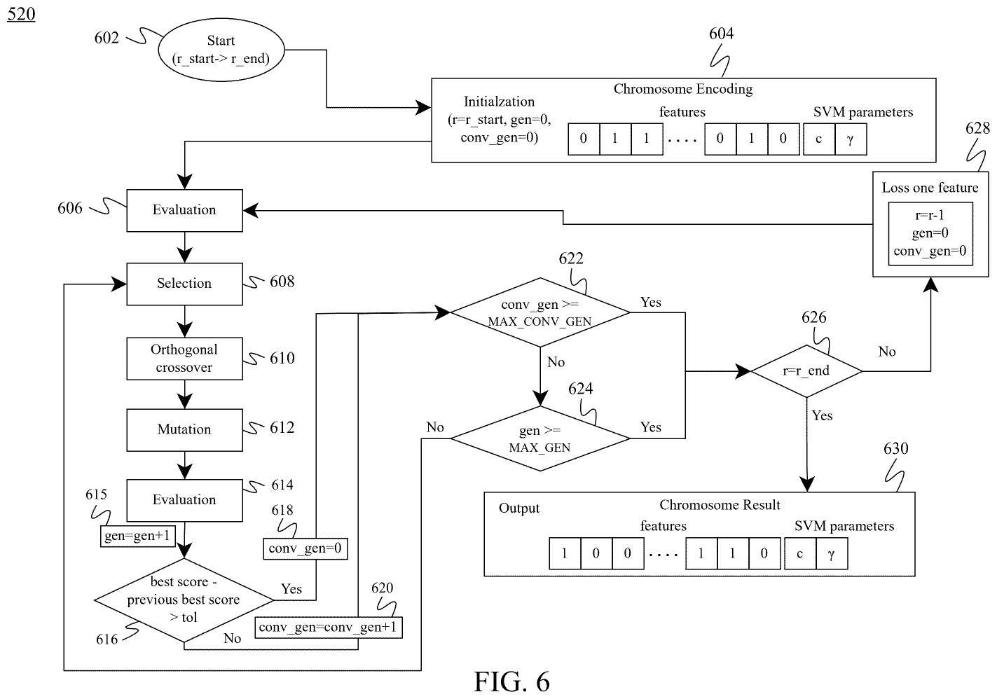

FIG. 6 is a flowchart of the procedure in block 520 in FIG. 5A. The procedure of the ICBGA with SVM in block 520 in FIG. 5A are shown in FIG. 6. For brevity, the steps of the ICBGA for predicting AKD are described. Steps of the ICBGA for predicting CKD and ESKD can be performed in a similar manner. In some embodiments, the input of the customized IBCGA algorithm is the phase-1 training dataset (e.g., first training subset in block 512 shown in FIG. 5A). The whole candidate dataset (e.g., obtained in block 502 in FIG. 5A) may be randomly divided into the training set (n_train) and test set (n_test) in a ratio of 8:2. The first training subset, which includes the AKI samples without missing values in the training set (n_train), may be used as the phase-1 training dataset of ELAKI for AKD (n=2312). Given that Npop=50, r_start=50, r_end=5, Pc=0.8, Pm=0.05, MAX_GEN=300, and MAX_CONV_GEN=30, fitness function of the ICBGA may be used to maximize accuracy in terms of five-fold cross-validation. Additionally, during chromosome encoding, the chromosome may include k binary genes fi for feature selection and two 4-bit genes for encoding parameters c and y of the SVM.

The customized IBCGA may include steps 1 to 8, namely, initialization, evaluation, selection, orthogonal crossover, mutation, termination test, inheritance, and outputting a signature, that are described as follows.

Step 1: (Initialization) Randomly generating a population of Npop individuals which consisting of r 1's and n-r 0's in the chromosome (block 604), where r=r_start, gen=0, and conv_gen=0.

Step 2. (Evaluation) Evaluating the fitness values of all individuals in the population (block 606).

Step 3. (Selection) Applying a tournament selection method to Npop pairs of individuals randomly selected to generate a mating pool of Npop individuals (block 608).

Step 4. (Orthogonal crossover) Selecting Pc-Npop parents from the mating pool to perform the orthogonal array crossover operation (block 610). The best two of all generated individuals and the parents are selected as the children of the crossover where Pc is the crossover probability.

Step 5. (Mutation) Applying a conventional mutation operation to the randomly selected Pm·Npop genes except the best individual where Pm is the mutation probability (block 612).

Step 6. (Termination test) Performing the evaluation step to identify the best individual Sr with the new best fitness value. Gen=gen+1. If there was no significant improvement compared with the previous best value in MAX_CONV_GEN generations or conv_gen=MAX_GEN, go to Step 7. Otherwise, conv_gen=conv_gen+1, go to Step 3.

Step 7. (Inheritance) Determining whether r is larger than r_end (block 626). If r >r_end, one bit in the binary genes fi for each individual is randomly changed from 1 to 0; r=r−1, gen=0, and conv_gen=0 (block 628); go to Step 2 (block 606).

Step 8. (Output) Let Sm be the best solution among the solutions Sr. Obtain a set of selected features (signature) and parameters c and y of SVM from decoding the chromosome of Sm (block 630).

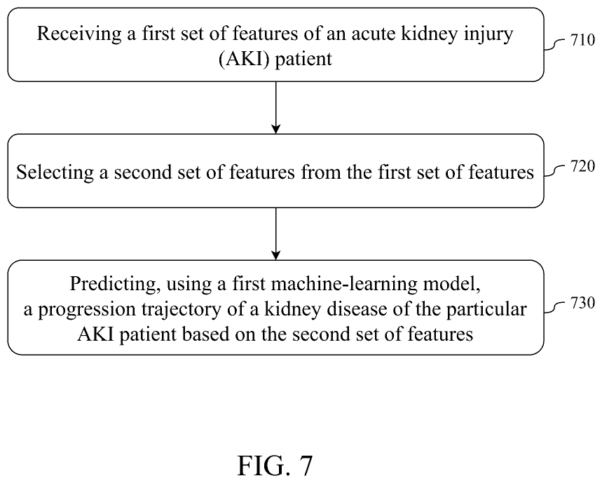

FIG. 7 is a flowchart of a method for predicting a progression trajectory from acute kidney injury (AKI) to a kidney disease. Please refer to FIGS. 5A-5B and FIG. 7. Method 700 may include steps 710 to 730.

Step 710: Receiving a first set of features of an acute kidney injury (AKI) patient. For example, the first set of features may include 55 candidate features of the AKI patients, as shown in Table 1, which may include AKI stage, demographic factors (age, sex, blood type [A, B, AB, and O], drug allergy, and critical illness), laboratory values (SCr, BUN, eGFR, Na, K, GPT, GOT, and white blood cell differential count [Neutrophil, Lymphocyte, Monocyte, Eosinophil, and Basophil]), and medication use history within one month prior to AKI occurrence. The first set of features may be regarded as a candidate dataset.

Step 720: Selecting a second set of features from the first set of features using a preset algorithm. For example, the preset algorithm may be an evolutionary learning method with support vector machine (SVM), called ELAKI. ELAKI may conduct a customized inheritable bi-objective combinatorial genetic algorithm (IBCGA) with an intelligent evolutionary algorithm. The details of the ELAKI can be referred to the embodiments of FIGS. 5A-5B, and those of the IBCGA can be referred to the embodiment of FIG. 6. For example, the ELAKI may determine 12, 15, and 16 features (e.g., can also be referred to as risk factors, signatures, or signature features) from the 55 candidate features for predicting AKD, CKD, and ESKD, respectively.

Step 730: Predicting, using a first machine-learning model, a progression trajectory of one or more kidney diseases of the AKI patient based on the second set of features. For example, the one or more kidney disease may be acute kidney disease (AKD), chronic kidney disease (CKD), and end stage kidney disease (ESKD), but the present disclosure is not limited thereto.

In some embodiments, the EL-AKD, EL-CKD, and EL-ESKD models built by the proposed ELAKI method of the present disclosure may have a better AUC (e.g., an area under the receiver operating characteristic curve) compared with the respective SVMs using p values for predicting AKD, CKD, and ESKD, as shown in Table 3.

TABLE 3

| | | |
|---|---|---|
| Model | Features | AUC |

| | | | |
|---|---|---|---|
| AKD | EL-AKD | 12 | 0.747 |
| | Pv-SVM | 12 | 0.679 |
| CKD | EL-CKD | 15 | 0.906 |
| | Pv-SVM | 15 | 0.810 |
| ESKD | EL-ESKD | 16 | 0.939 |
| | Pv-SVM | 16 | 0.913 |

EL-AKD, EL-CKD, and EL-ESKD are compared with the corresponding models using p-value to select the same number of top-ranked features with the methods SVM. AUC refers to area under the receiver operating characteristic curve.

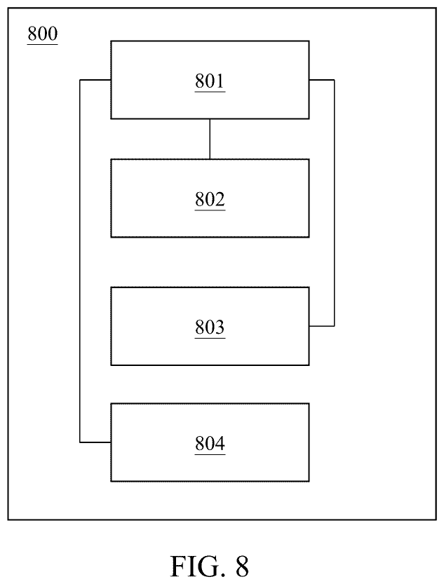

FIG. 8 is a schematic diagram showing a computer device 800 according to some embodiments of the present disclosure.

The computer device 800 may be capable of performing one or more procedures, operations, or methods of the present disclosure. The computer device 800 may be a server computer, a client computer, a personal computer (PC), a tablet PC, a set-top box (STB), a personal digital assistant (PDA), a cellular telephone, or a smartphone. The computing device 800 comprises processor 801, input/output interface 802, communication interface 803, and memory 804. The input/output interface 802 is coupled with the processor 801. The input/output interface 802 allows the user to manipulate the computing device 1100 to perform the procedures, operations, or methods of the present disclosure (e.g., the procedures, operations, or methods disclosed in FIGS. 5 to 7). The communication interface 803 is coupled with the processor 801. The communication interface 803 allows the computing device 800 to communicate with data outside the computing device 800, for example, receiving data including images or conditional features. A memory 804 may be a non-transitory computer readable storage medium. The memory 804 is coupled with the processor 801. The memory 804 has stored program instructions that can be executed by one or more processors (for example, the processor 801). In addition, the SVM models, EL-AKD, EL-CKD, and EL-ESKD models may be stored in the memory 804. Upon execution of the program instructions stored on the memory 804, the program instructions cause performance of the one or more procedures, operations, or methods disclosed in the present disclosure. For example, the program instructions may cause the computing device 800 to perform, for example, receiving a first set of features of an acute kidney injury (AKI) patient; selecting a second set of features from the first set of features using a preset algorithm; and predicting, using a first machine-learning model, a progression trajectory of one or more kidney diseases of the AKI patient based on the second set of features.

The scope of the present disclosure is not intended to be limited to the particular embodiments of the process, machine, manufacture, and composition of matter, means, methods, steps, and operations described in the specification. As those skilled in the art will readily appreciate from the disclosure of the present disclosure, processes, machines, manufacture, composition of matter, means, methods, steps, or operations presently existing or later to be developed that perform substantially the same function or achieve substantially the same result as the corresponding embodiments described herein may be utilized according to the present disclosure. Accordingly, the appended claims are intended to include within their scope processes, machines, manufacture, and compositions of matter, means, methods, steps, or operations. In addition, each claim constitutes a separate embodiment, and the combination of various claims and embodiments are within the scope of the disclosure.

The methods, processes, or operations according to embodiments of the present disclosure can also be implemented on a programmed processor. However, the controllers, flowcharts, and modules may also be implemented on a general purpose or special purpose computer, a programmed microprocessor or microcontroller and peripheral integrated circuit elements, an integrated circuit, a hardware electronic or logic circuit such as a discrete element circuit, a programmable logic device, or the like. In general, any device on which resides a finite state machine capable of implementing the flowcharts shown in the figures may be used to implement the processor functions of the present disclosure.

An alternative embodiment preferably implements the methods, processes, or operations according to embodiments of the present disclosure on a non-transitory, computer-readable storage medium storing computer programmable instructions. The instructions are preferably executed by computer-executable components preferably integrated with a network security system. The non-transitory, computer-readable storage medium may be stored on any suitable computer readable media such as RAMs, ROMs, flash memory, EEPROMs, optical storage devices (CD or DVD), hard drives, floppy drives, or any suitable device. The computer-executable component is preferably a processor, but the instructions may alternatively or additionally be executed by any suitable dedicated hardware device. For example, an embodiment of the present disclosure provides a non-transitory, computer-readable storage medium having computer programmable instructions stored therein.

While the present disclosure has been described with specific embodiments thereof, it is evident that many alternatives, modifications, and variations may be apparent to those skilled in the art. For example, various components of the embodiments may be interchanged, added, or substituted in the other embodiments. Also, all of the elements of each figure are not necessary for operation of the disclosed embodiments. For example, one of ordinary skill in the art of the disclosed embodiments would be able to make and use the teachings of the present disclosure by simply employing the elements of the independent claims. Accordingly, embodiments of the present disclosure as set forth herein are intended to be illustrative, not limiting. Various changes may be made without departing from the spirit and scope of the present disclosure. Even though numerous characteristics and advantages of the present disclosure have been set forth in the foregoing description, together with details of the structure and function of the invention, the disclosure is illustrative only. Changes may be made to details, especially in matters of shape, size, and arrangement of parts, within the principles of the invention to the full extent indicated by the broad general meaning of the terms in which the appended claims are expressed.

What is claimed is:

1. A method for predicting a progression trajectory from acute kidney injury (AKI) to a kidney disease, the method comprising:
receiving a first set of features of a particular acute kidney injury (AKI) patient;
selecting a second set of features from the first set of features using a preset algorithm; and
predicting, using a first machine-learning model, a progression trajectory of a kidney disease of the particular AKI patient based on the second set of features.

2. The method of claim 1, wherein the preset algorithm is a machine-learning feature-selection algorithm.

3. The method of claim 2, wherein the kidney disease comprises an acute kidney disease (AKD), a chronic kidney disease (CKD), or an end stage kidney disease (ESKD).

4. The method of claim 1, wherein the second set of feature include use of diuretics, use of antibiotics, and a value of creatinine.

5. The method of claim 1, wherein the first classification model comprises an evolutionary AKD SVM (EL-AKD) model, and the method further comprises: predicting, using the first machine-learning model, the progression trajectory of an acute kidney disease of the particular AKI patient based on the second set of features.

6. The method of claim 1, wherein the first classification model comprises an evolutionary CKD SVM (EL-CKD) model, and the method further comprises: predicting, using the first machine-learning model, the progression trajectory of a chronic kidney disease of the particular AKI patient based on the second set of features.

7. The method of claim 1, wherein the first classification model comprises an evolutionary ESKD SVM (EL-ESKD) model, and the method further comprises: predicting, using the first machine-learning model, the progression trajectory of an end stage kidney disease of the particular AKI patient based on the second set of features.

8. The method of claim 3, wherein a first training set extracted from a candidate dataset, which comprises a plurality of candidate features of a plurality of AKI samples, is used by the preset algorithm, and the first training set comprises the AKI samples with all of the candidate features.

9. The method of claim 8, further comprising:
determining a plurality of signature features of the kidney disease using the preset algorithm;
extracting a second training set, which comprises the determined signature features of the kidney disease, from the candidate dataset; and
training the first classification model using the second training set.

10. The method of claim 9, wherein the second set of features comprises the determined signature features of the kidney disease.

11. An apparatus for predicting a progression trajectory from acute kidney injury (AKI) to a kidney disease, the apparatus comprising:
at least one memory having computer executable instructions stored therein; and
at least one processor coupled to the at least one memory,
wherein the computer executable instructions cause the at least one processor to perform operations, and the operations comprise:
receiving a first set of features of a particular acute kidney injury (AKI) patient;
selecting a second set of features from the first set of features using a preset algorithm; and
predicting, using a first machine-learning model, a progression trajectory of a kidney disease of the particular AKI patient based on the second set of features.

12. The apparatus of claim 11, wherein the preset algorithm is a machine-learning feature-selection algorithm.

13. The apparatus of claim 12, wherein the kidney disease comprises an acute kidney disease (AKD), a chronic kidney disease (CKD), or an end stage kidney disease (ESKD).

14. The apparatus of claim 11, wherein the second set of feature include use of diuretics, use of antibiotics, and a value of creatinine.

15. The apparatus of claim 11, wherein the first classification model comprises an evolutionary AKD SVM (EL-AKD) model, and the operations further comprise: predicting, using the first machine-learning model, the progression trajectory of an acute kidney disease of the particular AKI patient based on the second set of features.

16. The apparatus of claim 11, wherein the first classification model comprises an evolutionary CKD SVM (EL-CKD) model, and the operations further comprise: predicting, using the first machine-learning model, the progression trajectory of a chronic kidney disease of the particular AKI patient based on the second set of features.

17. The apparatus of claim 11, wherein the first classification model comprises an evolutionary ESKD SVM (EL-ESKD) model, and the operations further comprise: predicting, using the first machine-learning model, the progression trajectory of an end stage kidney disease of the particular AKI patient based on the second set of features.

18. The apparatus of claim 13, wherein a first training set extracted from a candidate dataset, which comprises a plurality of candidate features of a plurality of AKI samples, is used by the preset algorithm, and the first training set comprises the AKI samples with all of the candidate features.

19. The apparatus of claim 18, wherein operations further comprises:
determining a plurality of signature features of the kidney disease using the preset algorithm;
extracting a second training set, which comprises the determined signature features of the kidney disease, from the candidate dataset; and
training the first classification model using the second training set.

20. The apparatus of claim 19, wherein the second set of features comprises the determined signature features of the kidney disease.
